(19) United States
(12) Patent Application Publication
(10) Pub. No.: US 2021/0030547 A1
(43) Pub. Date: Feb. 4, 2021

(54) CERAMIC SURFACE ARTHROPLASTY SYSTEM

(71) Applicant: IOANNIS VLACHOS, ATHENS (GR)

(72) Inventor: IOANNIS VLACHOS, ATHENS (GR)

(21) Appl. No.: 17/042,537

(22) PCT Filed: Apr. 2, 2019
(86) PCT No.: PCT/GR2019/000027

§ 371 Date: Sep. 28, 2020

(30) Foreign Application Priority Data
Apr. 4, 2018 (GR) 20180100146

(51) Int. Cl. A61F 2/36; A61F 2/30

(52) U.S. Cl. CPC A61F 2/3603; A61F 2002/30405; A61F 2002/3605; A61F 2002/30879; A61F 2002/3093; A61F 2002/30784; A61F 2310/00634; A61F 2310/0094; A61F 2310/00029; A61F 2310/00023; A61F 2310/00131; A61F 2310/00059; A61F 2002/30245; A61F 2002/3085; A61F 2/30749

(57) ABSTRACT

The invention relates to a ceramic surface arthroplasty system, which consists of a convex, hemispherical, metallic implant (1) and a hollow hemispherical metal implant (3). The implants are coated with porcelain or zirconium oxide layer on their friction surfaces to avoid the creation of metallic microparticles. The convex implant (1) has a navel (4) on the thread (6) of which a bone-piercing pre-tensioning needle (7) is screwed and is tightened with a washer (9) at the area of the major trochanter or bulge. Further, it has protruding ribs (5) immersed on the surface of the head.

### FIELD OF ART

The invention relates to the field of surgical instruments, devices or methods, and more particularly to artificial substitutes or replacements for parts of the body, and more particularly to implants for hip joint at the point of the femoral head, for replacement only of the epiphyseal sections of femur and acetabulum, without cutting off the entire natural femoral head.

### BACKGROUND OF THE INVENTION

The ceramic surface arthroplasty system disclosed in the present invention has not been disclosed in the prior art.

Under normal circumstances, the femoral head of a healthy human is articulated in the pelvic acetabulum, allowing a wide range of hip movements at multiple levels. Numerous conditions, such as osteoarthritis, can lead to disruption of joint function due to lesion of the articular cartilage and consequent pain, distortion and loss of functionality thereof. Total hip arthroplasty includes total replacement of the femoral head and the bone neck as well as the pelvic acetabulum, usually with metal alloy and polyethylene implants. This orthopaedic surgery is performed to reduce the intense pain that comes from the degenerated cartilage friction in order to restore functionality and improve the quality of life. The conventional method of total hip arthroplasty, with which the femoral head is removed, presents a number of drawbacks, mainly extensive bone loss and possible femoral implant detachment.

As an alternative and where feasible, it is proposed to maintain the head and neck of femur and replace only the articular surface of the head by proceeding to the so-called surface arthroplasty. This replacement is carried out to date using a shiny metallic implant, which is in the form of a spherical shell and is placed in the appropriately preformed head. The most important problem with this method is that the head implant is not strictly hemispherical but exceeds the equator by 20° or 30° on either side. As a result, there is a need to carve the bone and fill the gap with an acrylic polymer, removing a significant portion so that the implant can be placed. Considering that the forces exerted on the femoral head joints are great, weakening of the bone by reducing its resistance due to carving often causes a neck fracture or aseptic detachment of the implant.

Furthermore, the presently available implants have an internal centre axis up to 15 mm thick requiring a corresponding perforation of the femoral head and neck to penetrate therein. This results in an additional decrease in the area's resistance and decrease in the area of blood perfusion.

Additionally, the implants are restrained in the femoral head using acrylic polymer (cement), which is designed to connect the spongy bone of the head to the metal implant shell. During the polymerization phase, however, the exothermic reaction of this acrylic material further degrades the structural adequacy of the underlying spongy bone.

A more significant disadvantage may be the friction between the metal surfaces of the head and acetabular implants, which causes the detachment and migration of microparticles of chromium and cobalt ions, which create tumours locally and around arthroplasty. The ions are detected in circulating blood and potentially can cause organ tumours, such as in kidneys.

It is therefore an object of the present invention to advantageously overcome the aforementioned disadvantages and deficiencies of the prior art by proposing a ceramic surface arthroplasty system.

It is a further object of the present invention to provide a surface ceramic arthroplasty system which does not contribute to the generation of cobalt and chrome metal chips and ions during friction of the individual parts thereof.

A further advantage of the present invention is to provide a ceramic surface arthroplasty system in which both the implant placed in the acetabulum and the implant placed on the femoral head respectively are coated with porcelain or zirconium oxide layers on the parts coming into contact between them. The friction thereof generates only microparticles of calcium, zirconium and kaolin that are compatible with the human body.

A further object of the invention aiming at an increased functionality of the ceramic surface arthroplasty system is the presentation of an implant for the femoral head which carries a ‘navel’ at the pole in which a threaded metallic needle is screwed which intersects the femur and in outgoing of the major trochanter, by tightening a nut, applies pretension for initial stabilization.

It is a further object of the invention to provide a ceramic surface arthroplasty system, in which both the spherical shell of the acetabular implant and the corresponding spherical shell of the femoral head implant is strictly 180° to avoid peripheral bone removal leading to weakening of the area and to neck fracture over the time due to stress.

Another advantage of the invention is the presentation of a ceramic surface arthroplasty system, which does not require the use of acrylic polymer to attach the femur to the corresponding implant, since it carries meridian low thickness protruding ribs for greater postoperative rotation stability.

It is also an object of the present invention to provide a ceramic surface arthroplasty system in which the hollow inner surface of the implant is further covered with a porous metal layer which, over time, leads to permanent osteointegration in the underlying bone.

These and other objects, features and advantages of the invention will become apparent in the following detailed description.

### BRIEF DESCRIPTION OF THE DRAWINGS

The invention will become apparent to those skilled in the art with reference to the accompanying drawings, in which it is illustrated in an exemplary, non-limiting manner.

FIG. 1 shows a perspective illustration of the convex hemispherical metal implant, where the protruding ribs, the navel and a part of the metal needle are shown.

FIG. 2 shows a perspective illustration of the hollow, hemispherical, metallic implant.

FIG. 3 shows a perspective view of the femur with the implant applied to the femoral head, while FIG. 4 also shows in a perspective sketch the implant placed in the pelvic cavity.

FIG. 5 illustrates the ceramic surface arthroplasty system of the present invention in resetting the head into the pelvic acetabulum.

### DETAILED DESCRIPTION OF THE PREFERRED EMBODIMENT

Referring now to the accompanying drawings, illustrative embodiments of the ceramic surface arthroplasty system will be described in order to illustrate the advantages and particular features thereof.

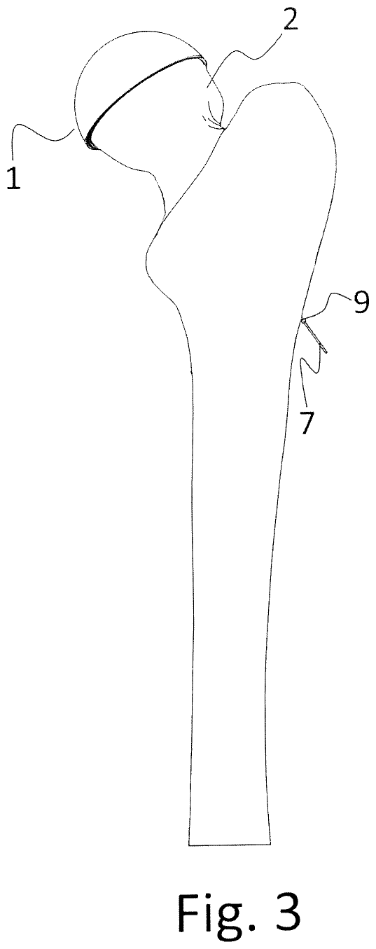

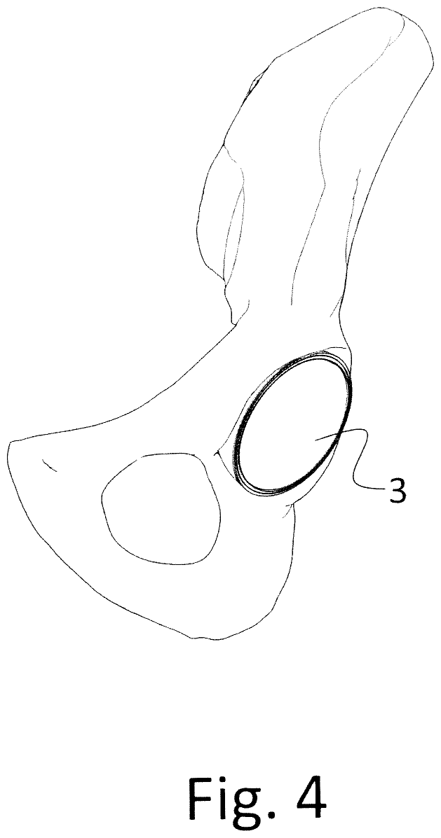

The present invention relates to a surface arthroplasty system, which consists of two metallic implants, a “female” and a “male” one. With respect to the femoral arthroplasty surface, the male “implant” is placed on the properly carved femoral head of the bone, FIG. 3, and it consists of a metallic convex, hemispherical surface covered with porcelain or zirconium oxide, while its internal concave surface, carries at the pole a navel into which a metallic threaded needle is screwed. Respectively, the “female” implant is placed in the pelvic acetabulum, suitably prepared by cartilage abrasion, FIG. 4, and consists of an outer convex, hemispherical metal surface whose internal concave surface is similarly covered with porcelain or zirconium oxide.

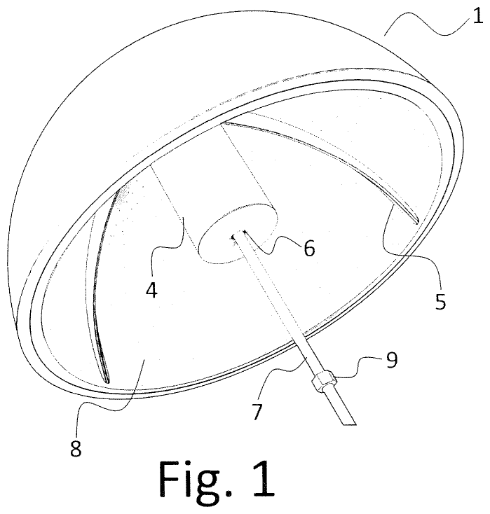

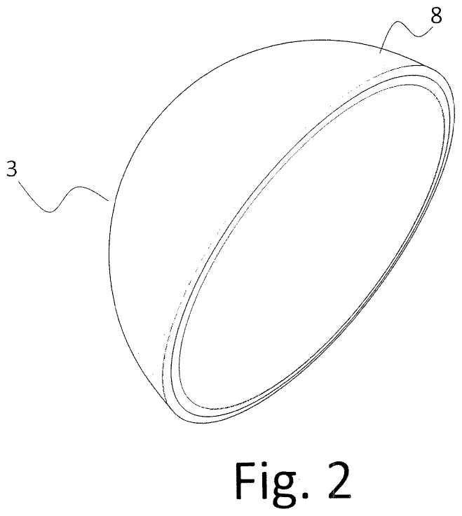

The convex, hemispherical metallic implant (1), FIG. 1, of the proposed system consists of a metal shell which is externally covered with a porcelain or zirconium oxide layer and is part of a thin metallic spherical shell of exactly 180°, forming substantially a hemisphere. Correspondingly, the internal configuration of the implant (1) is hemispherical. This has the beneficial effect that the circumference of the implant (1) socket is as wide as possible and a limited hemispherical carving is sufficient to place it in the femoral head (2), without significant bone removal. The hollow hemispherical metal implant (3) is respectively a portion of a thin metal spherical shell of exactly 180° whose the concave inner surface is covered with a porcelain or zirconium oxide layer and its convex outer surface coming into contact with the spongy bone of the acetabulum is covered with a porous metal layer (8) leading to permanent osteointegration in the underlying bone. The metals used in both the convex implant (1) and the concave implant (3) are, for example, titanium, cobalt, tantalum and chromium, or respective alloys thereof, as their biocompatibility allows for medical use. The thickness of each implant is never less than 1.5 mm and more usually ranges between 2 and 2.5 mm, depending on the anatomy of the individual patient.

In addition, the convex, hemispherical metallic implant (1) has internally a short ‘navel’ (4), which is located in the centre of the concave inner surface, on the so-called pole. This has the beneficial effect of not requiring extensive deep perforation of the head (2) and the femoral neck during implant placement, thus further preserving the integrity of the underlying bone and its perfusion.

Furthermore, on the inner surface of the convex, hemispherical, metallic implant (1) of the proposed ceramic surface arthroplasty system, there are meridian, thin protruding ribs (5). At the centre of the navel (4) there is a female thread (6) for receiving the pre-tensioning needle (7). With these two mechanisms, i.e. the ribs and pre-tensioning, sufficient early rotational and axial stability is ensured and no use of acrylic cement is required for attachment to the underlying bone. To ensure more stable implant stability, beyond the early one, the internal surface of the convex implant (1) is all covered with a porous metal layer (8), which, over time, leads to permanent osteointegration in the underlying bone.

The pre-tensioning needle (7) of the proposed implant, 2.5 mm thick, in part or in all of its length, has a thread corresponding to that of the navel (4). The one tip of the needle (7) is sharp. Thus, while one tip of the needle (7) can be screwed onto the navel (4) of the convex implant (1), its sharp tip may, after penetrating the head (2) and the femoral neck, according to surgeon's desirable direction, be fixed in the region of the major trochanter by means of the nut-washer combination (9), thus finally enforcing the desired pretension.

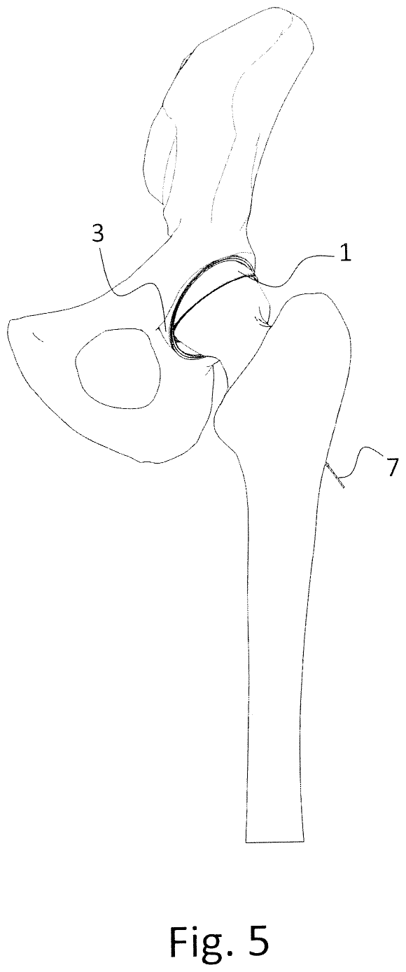

The application of the ceramic surface arthroplasty system in the surgical technique therefore requires perforation of the head (2) and the neck by using a needle (7) in a neck-femur direction tangential to the major trochanter and according to the desired axis, as it results from the preoperative design. Centrally the needle (7) remains sufficiently protruding from the femoral head while circumferentially emerging from the major trochanter. The pretensioning needle (7) remains in place and is used as a guide for the slightly hemispherical carving of the femoral head (2) and the creation of the short navel (4) socket, according to the size of the proposed convex, hemispherical, metallic implant (1). Then the implant (1) is screwed onto the needle (7) and is pulled from the side of the major trochanter. The nut/washer combination (9) is placed on the circumferentially protruding part of the needle (7) and up to the cortex of the major trochanter. The needle (7) portion that is left over the nut (9) after the latter has been tightened in the area of the major trochanter is cut off. The nut (9) tightening results in the precipitation of the implant (1) on the femoral head (2), the immersion of the ribs (5) of the implant (1) on the surface of the head (2) and finally in the creation of the desired pretensioning along the needle (7). Furthermore, the acetabulum is carved by a convex scraper to the desired size, corresponding to the convex implant (1), and transverberation of the hollow implant (3). Finally, the femoral head (2) is reset so that the glazed porcelain or zirconium oxide surface of the convex implant (1) is brought into contact with the concave surface of the hollow implant (3), which internally on its concave surface is covered by a porcelain or zirconium oxide layer, FIG. 5.

It is to be noted here that the description of the invention has been made by reference to exemplary embodiments, not being limited thereto. Any alteration or modification in terms of dimensions, morphology, used materials and components of manufacture and assembly and the stages of application of the procedure, if they are not a novel inventive step and do not contribute to the technical development of the already known one, are considered to be included in the purposes and contemplation of the present invention.

1. A ceramic surface arthroplasty system, consisting of a convex, hemispherical metallic implant (1) with a metal surface, on the inner surface of which there are meridian protruding ribs (5) and a navel (4) at the implant pole (1), with a female thread (6) on which a pretensioning needle (7) is screwed, which intersects the neck of the bone and is tightened in the area of the major trochanter or bulge using a nut-washer (9) set, and a hollow hemispherical metal implant (3) with a metal surface, characterized in that both implants are coated with porcelain or zirconium oxide on the adjacent surfaces.

2. A ceramic surface arthroplasty system, according to claim 1, characterized in that the inner side of the convex hemispherical metal implant (1) and the outer side of the hollow hemispherical metal implant (3) are covered with a porous metal layer (8) to achieve osteointegration with the corresponding bones.
